(19) United States
(12) Patent Application Publication
(10) Pub. No.: US 2016/0160179 A1
(43) Pub. Date: Jun. 9, 2016

(54) METHOD FOR OBTAINING A POPULATION OF ISOLATED BRITE ADIPOCYTES

(71) Applicants: INSERM (INSTITUT NATIONAL DE LA SANTÉ ET DE LA RECHERCHE MÉDICALE), Paris (FR); UNIVERSITE PAUL SABATIER TOULOUSE III, Toulouse (FR)

(72) Inventors: Jean GALITZKY, Toulouse (FR); David ESTEVE, Toulouse (FR); Anne BOULOUMIE, Toulouse (FR)

(21) Appl. No.: 14/904,149

(22) PCT Filed: Jul. 10, 2014
(86) PCT No.: PCT/EP2014/064778

§ 371 Date: Jan. 11, 2016

(30) Foreign Application Priority Data
Jul. 11, 2013 (EP) 13305988.1

(51) Int. Cl. C12N 5/077; A61K 35/35; G01N 33/50

(52) U.S. Cl. CPC C12N 5/0653; G01N 33/5044; A61K 35/35; C12N 2501/155

(57) ABSTRACT

The present invention relates to a method for obtaining a population of white/brite preadipocytes from precursor cells comprising the step of selecting cells for the expression of mesenchymal stem cell antigen 1 (MSCA1). The invention also relates to a method for obtaining brite adipocytes.

### FIELD OF THE INVENTION

The invention relates to a method for obtaining a population of isolated brite adipocytes from precursor cells.

### BACKGROUND OF THE INVENTION

Obesity, as described by WHO, is <<an abnormal or excessive fat accumulation that presents a risk to health”. Its prevalence differs drastically from one country to another, affecting more than 30% of the population of the United States, and is in constant expansion. Given its cost, on both economy and public health, many are trying to find a way to cure or to slow down what can be described as one of the world's leading preventable causes of death. The underlying process responsible for said fat accumulation is adipogenesis.

Adipogenesis is defined as the process by which adipocytes are formed. External stimuli regulate networks of transcription factors and second messengers prompt mesenchymal stem/progenitor cells to commit to the adipocyte lineage. These cells become preadipocytes that can subsequently undergo a terminal differentiation event and express adipocyte-specific genes and functions (Cristancho and Lazar, 2011) (Tang and Lane, 2012). When considering this process as linear and unidirectional in time, cells will lose their self-renewal capacities and will restrain their differentiation potentials (Gesta et al., 2007). Determination of the exact state of differentiation of human adipocytes is hindered due to a lack of markers, which can identify non-committed stem/progenitor cells, committed progenitor cells or preadipocytes. While Pref1 (Smas and Sul, 1993) and ZFP423 (Gupta et al., 2010) have been identified as murine markers for committed preadipocytes, similar markers for human progenitor cells and preadipocytes have not been determined.

The existence of adipogenesis in adult adipose tissues (AT) remains to be clearly demonstrated. However, several observations suggest that adipogenesis is taking place in adult AT. Adipogenesis might play an important role in the physiological turnover of adipocytes, reported to be approximately 10% every year in human subcutaneous AT (Spalding et al., 2008). Anatomic location-dependent differences in the adipogenic potential of murine adipocyte progenitor cells has been reported (Macotela et al., 2012). Indeed, a reduced adipogenic capacity was observed in visceral AT when compared to subcutaneous AT. Intrinsic differences between progenitor cells from different AT depots may contribute to previously reported differences in adipocyte function and turnover in rodents (Gesta et al., 2007). Modulation of adipogenesis has been suspected to occur in mice as a result of obesity and associated pathologies (Wang et al., 2008). It is important to resolve whether or not depot-specific differences in progenitor cells also exist for human adipose tissues, which exhibit clear depot-specific functionality (Lafontan and Girard, 2008).

The human cell models to study adipogenesis (white or brite) that are available present limits. Human adipose stromal cells (hASCs) are generally obtained through ex vivo expansion of human adipose tissue whole stroma-vascular cells selected by plastic adhesion and passaging. Since no prior selections are performed, endothelial cells, smooth muscle cells and progenitor cells as well as fibroblasts, all capable to adhere to plastic dishes and to proliferate, will be present. Moreover passaging of cells will modify the phenotype of the cells. hMADS are cell lines originating from young adipose tissue donors (less than seven years old). They are also selected from whole stroma-vascular cells by their capacity to adhere and to proliferate in specific medium (containing fibroblast growth factor).

During the last few years, comparisons between white and brown AT in mice have led to evidence supporting the concept that both white and brown adipocytes have distinct lineages (Timmons et al., 2007). The brown adipocyte originates from a progenitor cell common to the myocyte lineage and characterized by the expression of MYF5 (Seale et al., 2008). Recently, a third type of fully differentiated adipocyte has emerged: the brown in white adipocyte, which has been defined as brite (Petrovic et al., 2010) or beige (Ishibashi and Seale, 2010). It shares a subset of features common to brown adipocytes such as the expression of uncoupling protein 1 (UCP1). Brite/beige adipocytes appear in mice in some “classical” white AT depots, particularly in the inguinal depot, under long-term PPARy agonist treatment (Petrovic et al., 2010) or beta-3 adrenergic receptor stimulation (Seale et al., 2008). Such conditions have been associated with resistance to diet-induced obesity and metabolic alterations. Therefore, it is strongly suggested that stimulation of brite/beige adipocytes might be a strategy to limit obesity-associated pathologies (Cinti, 2011). No data are available in humans concerning the presence and the role played by brite/beige adipocytes in the distinct white AT locations. Whether white and brite/beige adipocytes share common progenitor cells (Lee et al., 2012) and/or originate from the transdifferentiation of white adipocytes into brown-like adipocytes (Barbatelli et al., 2010) remains to be clearly established. However, in mice, as well as in humans, specific brite/beige gene expression profiles have been described, permitting one to trace the brite/beige potential of progenitor cell subsets (Gburcik et al., 2012; Seale et al., 2011).

It has previously been demonstrated in human adult AT that native CD34+/CD31− cells exhibit progenitor cell properties such as their ability to proliferate and express angiogenic (Miranville et al., 2004) and adipogenic potentials (Sengenes et al., 2005). Moreover, they expressed at different levels several mesenchymal stem/stromal cell markers and localized both at perivascular and stromal positions (Maumus et al., 2011). Such a progenitor cell population appears quite similar to that identified in murine models (Rodeheffer et al., 2008) (Tang et al., 2008).

However, there remains an unmet need in the art for markers for the common white/brite preadipocyte.

### SUMMARY OF THE INVENTION

The inventors have found that mesenchymal stem cell antigen 1 (MSCA1) was a marker for the white and brite/beige preadipocytes and show that this marker can used to isolate the common precursors in order to obtain a population of isolated brite adipocytes.

Thus, the present invention relates to a method for obtaining a population of white/brite preadipocytes from precursor cells comprising the step of selecting cells for the expression of mesenchymal stem cell antigen 1 (MSCA1).

The invention also relates to an isolated population of white/brite preadipocytes obtainable by the method described above.

The invention also relates to the use of a population of white/brite preadipocytes obtainable by the method described above in a method of screening.

The invention also relates to a method for screening drugs useful for treating obesity comprising:
obtaining a population of white/brite preadipocytes according to the method defined above;
optionally immortalizing said cells;
incubating said cells in the presence of a test compound;
measuring the adipogenesis.

The invention also relates to a method for obtaining a population of isolated brite adipocytes from precursor cells comprising the steps consisting of:
selecting cells for the expression of MSCA1;
culturing the MSCA1-positive cells in the presence of an agent that induces brite differentiation.

In another aspect, the invention relates to the use of MSCA1 as a biomarker.

In particular, the invention relates to the use of MSCA1 as a biomarker for brite/white preadipocytes.

### DETAILED DESCRIPTION OF THE INVENTION

### Method for Obtaining a Population of White/Brite Preadipocytes Adipocytes

The present invention relates to a method for obtaining a population of white/brite preadipocytes from precursor cells comprising the step of selecting cells for the expression of mesenchymal stem cell antigen 1 (MSCA1).

Indeed, the inventors have found that MSCA1 is a marker for the common precursor cell population which can lead to both white adipocytes and brite/beige adipocytes. Once these cells have been selected, differentiation can be induced towards one or the other of these two pathways.

In one aspect, the invention relates to the use of MSCA1 as a biomarker.

In particular, the invention relates to the use of MSCA1 as a biomarker for brite/white preadipocytes.

As used herein, the expression “white/brite preadipocyte” or “brite/white preadipocyte” refers to a cell that can differentiate either into a white adipocyte or into a brite adipocyte.

As used herein the expressions “brite adipocyte”, “brown-in-white adipocyte” and “beige adipocyte” are used interchangeably and have the general meaning in the art.

Brite adipocytes are adipocytes that are generally present in the white adipose tissue but that phenotypically resemble brown adipocytes. In particular, brite adipocytes express the “thermogenin” protein or “uncoupled protein-1” (UCP1). Thermogenin is an uncoupling protein found in the mitochondria of brown adipose tissue (BAT). It is used to generate heat by non-shivering thermogenesis. Non-shivering thermogenesis is the primary means of heat generation in hibernating mammals and in human infants.

As used herein, the expression “precursor cells” refers to cells that are not fully differentiated or committed towards a given cell lineage. Precursor cells can include mesenchymal stem cells, which are multipotent, as well as committed progenitor cells.

In one embodiment of the invention, the precursor cells useful as starting material for carrying out the present invention are obtained from adipose tissue, preferably from the stroma-vascular fraction (SVF) cells of adipose tissues.

Any method suitable for providing the SVF cells can be used.

Typically, SVF cells can be obtained from adipose tissue by subjecting said adipose tissue to enzymatic digestion, filtration and centrifugation steps.

In a preferred embodiment, the precursor cells are obtained as follows:
incubation of adipose tissue with dispase and collagenase under conditions allowing for suitable digestion of said adipose tissue;
filtration of the digestate using a 250 μm filter;
centrifugation at 400 g;
collection of the pellet and treatment with erythrocyte lysis buffer (155 mmol/L NH4CL; 5.7 mmol/L K2HPO4; 0.1 mmol/L EDTA; pH 7.3);
successive filtrations with 100 μm, 70 μm and 40 μm meshes in order to remove matrix fragments.

The SVF cells comprise preadipocytes, mesenchymal stem cells (MSC), endothelial progenitor cell, T cells, B cells, mast cells as well as adipose tissue macrophages.

The inventors have found that this mixed population of cells comprises cells that can be differentiated into brite adipocytes, and that these cells express MSCA1.

In one embodiment, the method of the invention further comprises a step of selecting the total progenitor population from the SVF cells, prior to selecting cells for the expression of MSCA1.

In one embodiment, immune cells are removed from the SVF cells using CD45-depletion.

In one embodiment, endothelial cells are removed using CD31 depletion.

In one embodiment, CD34-positive cells are selected.

Hence, in one aspect, the invention relates to a method for obtaining a population of white/brite preadipocytes comprising the steps of:
obtaining the SVF cells from adipose tissue;
selecting the CD45−/CD31−/CD34+ cells;
selecting cells for the expression of MSCA1.

In another embodiment of the invention, the precursor cells are mesenchymal stem cells from human adipose tissue (hMADS) as described in Rodriguez et al., 2004.

As used herein, the term “MSCA1” or “mesenchymal stem cell antigen 1”, also known as “tissue non-specific alkaline phosphatase”, is a human protein expressed on the cell membrane of certain mesenchymal stem cells.

It is encoded by the MSCA1 gene and can be found under the OMIM reference number 171760.

The step of selecting cells for the expression of MSCA1 can be carried out according to any suitable method known in the art.

In one embodiment, the step of selecting cells for the expression of MSCA1 is carried out using immunoselection methods, such as immunobeads or Fluorescence-Activated Cell Sorting (FACS).

Typically, the step of selecting cells for the expression of MSCA1 can be carried out using the W8B2 antibody described in Vogel et al., 2003 (Haematologica 88:126-133).

As used herein, the expressions “immunobeads” or “magnetic beads” or “microbeads” are used interchangeably. They refer to microscopic magnetic beads that are coated with an antibody specific for a given antigen. Cell suspensions are incubated with immunobeads and placed in a magnetic column or magnet. Cells expressing the given antigen on their cell surface are retained in the column or magnet, whereas cells that do not express said antigen are eluted.

Typically, the step of selecting cells for the expression of MSCA1 can be performed using the Anti-MSCA MicroBead kit commercialized by Miltenyi Biotech under reference 130-093-583. Alternatively, antibodies (Clone B4-78 or clone W8B2) directed against human alkaline phosphatase from R&D system (Ref FAB 1448A when coupled to APC) or from Biolegend (reference 327-306 when coupled to PE for 100 tests) or Miltenyi Biotech (130-093-587) coupled to fluorochromes can be combined to Anti-fluorochrome microbead kit commercialized by Miltenyi Biotech (under reference 130-090-855 for APC for example).

Cell sorting protocols using fluorescent labeled antibodies directed against MSCA1 (Miltenyi Biotech) or alkaline phosphatase (clone W8B2 Biolegend or clone B4-78 (R&D system) in combination with antibodies directed against CD34 and CD31 coupled with distinct fluorochromes can allow direct sorting, using cell sorters with the adequate optic configuration, of the CD34+/CD31−/MSCA1+ cells.

In one aspect, the invention relates to an isolated population of brite/white preadipocytes obtainable according to the method described above.

The isolated population of brite/white preadipocytes is preferably an isolated population of human white/brite preadipocytes.

In a preferred embodiment, the isolated brite/white preadipocytes are native. They are primary non passaged cells.

In addition to be positive for MSCA1, the isolated brite/white preadipocytes obtainable according the method of the invention may be also positive for the marker(s) CD34 and/or CD271.

The isolated brite/white preadipocytes may also be negative for CD45 and/or CD31.

Therefore, in a preferred embodiment, the isolated brite/white preadipocytes are CD45−/CD34+/CD31−/CD271+/MSCA1+.

The method of the invention makes it possible to obtain a population of brite/white preadipocytes with a high purity.

Therefore, in one embodiment, the isolated is a substantially pure homogenous population of CD45−/CD34+/CD31−/CD271+/MSCA1+ brite/white preadipocytes.

The term “substantially pure homogenous population”, as used herein, refers to a population of cells wherein the majority (e.g., at least about 70%, preferably at least about 80%, more preferably at least about 90%, most preferably at least about 95%) of the total number of cells have the specified characteristics of the CD45−/CD34+/CD31−/CD271+/MSCA1+ brite/white preadipocytes of interest.

In one embodiment of the invention, the isolated population of brite/white preadipoctyes can be immortalized. Any method, known to the person skilled in the art, for immortalizing cell lines can be used. Suitable methods include, but are not limited to, lentiviral transduction using antigen T of SV40 (pLenti-SV40-T), human telomerase reverse transcriptase protein (pLenti-hTERT), followed by serial passaging.

The brite/white preadipocytes (or the substantially pure homogenous population of brite/white preadipocytes) of the invention can be used for several types of applications, which include, but are not limited to:
study of the mechanisms involved in adipogenesis
study of the mechanisms involved in white adipogenesis
study of the mechanisms involved in brite adipogenesis
use in cosmetic surgery (e.g. reinjection by heterologous grafts of white adipocytes in order to reconstruct tissue).

### Screening Methods of the Invention

In one aspect, the invention provides a method for screening compounds that induce differentiation into brite adipocytes comprising the steps of:
incubating a population of white/brite preadipoctyes or a population of immortalized white/brite preadipocytes as described above in the presence of a test compound;
assessing the differentiation into brite adipocytes.

Typically, a test compound will be deemed to induce differentiation into brite adipocytes if the number of brite adipocytes obtained after incubation with said test compound is higher than the number of brite adipocytes obtained after incubation in the absence of said test compound (control conditions).

Methods for assessing the differentiation into brite adipocytes are known in the art. Typically, direct immunohistochemical analyses using antibodies directed against UCP1 can be performed. alternatively, gene reporter assays using transduction or transfection of cells with fluorescent and/or luminescent reporter gene under the control of the human UCP1 promoter can be used.

### Differentiation into Brite Adipocytes

The inventors have found that cells expressing MSCA1 exhibit a high capacity to differentiate into either white or brite adipocytes.

In one embodiment of the invention, the method of the invention further comprises a differentiation step.

Typically, the differentiation step comprises culturing the MCSA1+ cells in the presence of an agent that induces adipogenesis.

As used herein, the expression “agent that induces adipogenesis” refers to an agent that is capable of inducing the differentiation of preadipocytes into adipocytes.

Agents that induce adipogenesis have already been described in the art.

Suitable agents comprise, but are not limited to, non-selective phosphodiesterase (PDE) inhibitors, beta-adrenergic agonists, Bone Morphogenetic Proteins (BMPs), and thiazolidinediones.

Non-selective PDE inhibitors are drugs that block two or more of the five subtypes of the enzyme phosphodiesterase (PDE), thereby preventing the inactivation of the second messengers cyclic AMP and/or cyclic GMP.

Non-selective PDE inhibitors include, but are not limited to: caffeine, aminophylline, 3-isobutyl-1-methylxanthine (IBMX), paraxanthine, pentoxifylline, theobromine and theophylline.

Beta-adrenergic agonists are compounds that activate the adrenaline (also known as epinephrine) beta receptors.

The beta-adrenergic agonists suitable for carrying out the present invention include, but are not limited to, adrenalin, noradrenaline, T0509 compounds, salbutamol and procaterol, BRL37314, dobutamine, terbutaline, isoproterenol, CGP12177A and CL316243.

Bone Morphogenetic Proteins (BMPs) are a group of growth factors also known as cytokines and as metabologens. Originally discovered by their ability to induce the formation of bone and cartilage, BMPs are now considered to constitute a group of pivotal morphogenetic signals, orchestrating tissue architecture throughout the body.

Recently, BMP7 was shown to be involved in the genesis of brown adipose tissue (BAT) in mice.

Thiazolidinediones are agents that stimulate the PPARgamma receptor. They include, but are not limited to rosiglitazone, pioglitazone and troglitazone.

In a preferred embodiment of the invention, the agent that induces adipogenesis is selected from the group consisting of IBMX, BMP7 and rosiglitazone.

In a preferred embodiment, said agent that induces adipogenesis is an agent that induces brite adipogenesis, i.e. an agent that induces differentiation into brite adipocytes.

As used herein, the expression “agent that induces brite adipogenesis” refers to an agent that is capable of inducing the differentiation of preadipocytes into brite adipocytes. Typically, an agent is deemed to induce brite adipogenesis if it leads to an increase in the expression of the gene encoding UCP1.

The skilled person in the art can readily design suitable assays for assessing the increase in the expression of the gene encoding UCP1. Typically, the expression level of the gene encoding UCP1 can be assessed by performing quantitative RT-PCR on the mRNA under different conditions (before and after culture in the presence of the agent). Typically, direct immunohistochemical analyses using antibodies directed against UCP1 can be performed. Alternatively, gene reporter assays using transduction or transfection of cells with fluorescent and/or luminescent reporter gene under the control of the human UCP1 promoter can be used.

In a preferred embodiment, said agent that induces brite adipogenesis is selected from the group consisting of IBMX, BMP7 and mixtures thereof.

Further agents that induce brite adipogenesis can be identified by the screening method describes above.

Typically, the method of the invention comprises the step of culturing the white/brite preadipocytes obtained by selecting MSCA1-positive cells in the presence of IBMX for a period of time suitable for inducing differentiation into brite adipocytes.

Typically, the period of time suitable for inducing differentiation into brite adipocytes can vary between 3 and 15 days, preferably between 5 and 12 days, even more preferably about 10 days.

The concentration of IMBX in the culture medium suitable for the method of the invention is between 0.10 mM and 1 mM, preferably between 0.15 mM and 0.5 mM, even more preferably about 0.25 mM.

### Population of Isolated Brite Adipocytes of the Invention and Uses Thereof

A further object of the invention relates to an isolated population of brite adipocytes obtainable by a method as above described.

As used herein, the term “isolated” refers to a cell or a population of cells which has been separated from at least some components of its natural environment.

The isolated population of brite preadipocytes is preferably an isolated population of human brite preadipocytes.

In a preferred embodiment, the isolated brite preadipocytes are native. They are primary non passaged cells.

In addition to be positive for MSCA1, the isolated brite preadipocytes obtainable according the method of the invention may be also positive for the marker(s) CD34 and/or CD271.

The isolated brite preadipocytes may also be negative for CD45 and/or CD31.

Therefore, in a preferred embodiment, the isolated brite preadipocytes are CD45−/CD34+/CD31−/CD271+/MSCA1+.

The method of the invention makes it possible to obtain a population of brite preadipocytes with a high purity.

A further object of the invention relates to an isolated substantially pure homogenous population of brite adipocytes obtainable by a method as above described.

The term “substantially pure homogenous population”, as used herein, refers to a population of cells wherein the majority (e.g., at least about 70%, preferably at least about 80%, more preferably at least about 90%, most preferably at least about 95%) of the total number of cells have the specified characteristics of the brite adipocytes of interest.

In one embodiment, the isolated is a substantially pure homogenous population of CD45−/CD34+/CD31−/CD271+/MSCA1+ brite preadipocytes.

The brite adipocytes (or the substantially pure homogenous population of brite adipocytes) of the invention can be used for several types of applications, which include, but are not limited to:
treatment of obesity
study of the mechanisms involved in thermogenesis.

Therefore the invention relates to a pharmaceutical composition comprising a substantially pure homogenous population of brite adipocytes of the invention and optionally a pharmaceutically acceptable carrier or excipient. In certain embodiments, a pharmaceutical composition may further comprise at least one biologically active substance or bioactive factor.

As used herein, the term “pharmaceutically acceptable carrier or excipient” refers to a carrier medium which does not interfere with the effectiveness of the biological activity of the progenitor cells, and which is not excessively toxic to the host at the concentrations at which it is administered. Examples of suitable pharmaceutically acceptable carriers or excipients include, but are not limited to, water, salt solution (e.g., Ringer's solution), oils, gelatines, carbohydrates (e.g., lactose, amylase or starch), fatty acid esters, hydroxymethylcellulose, and polyvinyl pyroline. Pharmaceutical compositions may be formulated as liquids, semi-liquids (e.g., gels) or solids (e.g., matrix, lattices, scaffolds, and the like).

As used herein the term “biologically active substance or bioactive factor” refers to any molecule or compound whose presence in a pharmaceutical composition of the invention is beneficial to the subject receiving the composition. As will be acknowledged by one skilled in the art, biologically active substances or bioactive factors suitable for use in the practice of the present invention may be found in a wide variety of families of bioactive molecules and compounds. For example, a biologically active substance or bioactive factor useful in the context of the present invention may be selected from anti-inflammatory agents, anti- apoptotic agents, immunosuppressive or immunomodulatory agents, antioxidants, growth factors, and drugs.

A related aspect of the invention relates to a method for treating a subject suffering from obesity, said method comprising a step of administering to the subject an efficient amount of a substantially pure homogenous population of brite adipocytes of the invention (or a pharmaceutical composition thereof).

As used herein, the term “subject” refers to a mammal, preferably a human being.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “efficient amount” refers to any amount of a substantially pure homogenous population of brite adipocytes (or a pharmaceutical composition thereof) that is sufficient to achieve the intended purpose.

Without wishing to be bound by theory, it is believed that the brite adipocytes of the invention or the pharmaceutical composition thereof, after administration by injection into the adipose tissue of the subject, will carry out thermogenesis, resulting in the reduction of the adipose tissue.

The present invention also relates to a method for screening drugs useful for treating obesity comprising:
obtaining a population of brite preadipocytes according to the method as described above,
incubating said cells in the presence of a test compound,
assessing adipogenesis.

### EXAMPLE

The invention will be further illustrated by the following examples. However, these examples should not be interpreted in any way as limiting the scope of the present invention.

### Materials and Methods

Adipose tissue collection

Subcutaneous human adipose tissues (ATs) were obtained from healthy adult women undergoing plastic surgery for fat removal for aesthetic purposes (n=54, mean body mass index (BMI) 28±5.6 (21.3-39.3), mean age 43.1±11.9 (26-69)). Subcutaneous and visceral matched AT biopsies were obtained from obese patients undergoing gastric bypass surgery (ClinicalTrials.gov Identifier: NCT01525472). All donors gave their informed consent. Inguinal and epididymal AT were obtained from male 20-22 week-old C57B16 mice. Fat collection procedures were approved by local ethical committees.

Isolation of adipose tissue stroma-vascular cells

AT was cut in small pieces. After dispase (2.4 U/ml in phosphate-buffered saline (PBS), pH 7.4, volume/volume) and collagenase (250 U/ml in PBS, 2% bovine serum albumin (BSA), pH 7.4, volume/volume) digestion for 30 minutes at 37° C. for human AT or collagenase digestion only for mice AT, the cell suspension was filtered with 250 μm filter. The stroma-vascular fraction (SVF) was obtained after 400 g centrifugation at room temperature and treatment with Erythrocyte Lysis Buffer (155 mmol/L NH4Cl; 5.7 mmol/L K2HPO4; 0.1 mmol/L ethylenediaminetetraacid (EDTA); pH 7.3) for 10 min. Finally, matrix fragments were removed using successive filtrations through 100 μm, 70 μm, and 40 μm nylon meshes. Viable SVF cells were counted using trypan blue exclusion method and Neubauer chamber.

Adipocyte number and diameter

Adipocytes obtained after collagenase digestion were centrifuged at 100 g for 2 min. 20 μl of adipocytes were fixed in 380 μl CellFix (BD Biosciences (le Pont de Claix, France)) for 1 hour at 37° C. Three phase contrast microscopy images (Nikon Eclipse TE300) were taken and adipocyte diameter and number were determined using ImageJ software.

Isolation of cell subsets using immunoselection/depletion with magnetic nanobeads

An immuno-selection/depletion approach was developed to obtain the native progenitor cell subsets starting from 50.10⁶ to 150.10⁶ SVF cells. The total progenitor cell population (CD34+/CD31− cells) was obtained in two steps: immune cells were first removed using CD45 positive cell-depletion step (CD45-depletion kit from Stemcell Technologies, Grenoble, France) followed by endothelial cell depletion step using an anti-CD31-depletion kit (R&D Systems, Lille, France) according to manufacturer's protocol. To further isolate the distinct progenitor cell subsets, the Mscal-positive cells were selected from the CD45−/CD31− cells, the −/CD271 positive cells were selected from the CD45−/CD31−/MSCA1− cells and the CD45−/CD31−/MSCA1−/CD271− (−/−) cells using CD34 immuno-magnetic selection kits (MSCA1+, CD271+ and CD34+ immuno-magnetic selection kits were from Miltenyi Biotec, Paris, France). The purity of each fraction was assessed after selection by flow cytometry analyses.

The purity of the population of CD45−/CD34+/CD31−/CD271+/MSCA1+ cells is 80% +/−10% when cells are sorted with immune-magnetic beads.

The population of CD45−/CD34+/CD31−/CD271+/MSCA1+ cells may also be sorted by cytometry. In this case, the purity of the population of CD45−/CD34+/CD31−/CD271+/MSCA1+ cells is 95% +/−1%.

Flow cytometry analyses

10⁵ cells in 100 μl FACS Buffer were incubated with fluorescent-labeled monoclonal antibodies or respective isotype controls ( 1/20 diluted, 30 min, 4° C.). Mitochondrial content was assessed using Mitotracker® green FM (25 nmol/L) in FACS Buffer for 30 min at 37° C. before cell surface staining. After washing steps, the cells were analyzed by flow cytometry using a FACS Canto™ II flow cytometer and Diva Pro software (BD Biosciences).

In vitro adipogenesis

120 000 cells were plated per cm² in growth medium (ECGM MV) supplemented with 50 mg/ml penicillin-streptomycin. 48 h after seeding, medium was changed into basal defined medium (ECBM) supplemented with 50 mg/ml penicillin-streptomycin, 66 nmol/L Insulin, 1 nmol/L Triiodothyronine, 0.1 μg/ml human Transferrin, 100 nmol/L Cortisol (ITTC, Control) supplemented or not with 3 μmol/L Rosiglitazone or 0.25 mM isobuthylmethaxantine (IBMX). After 3 days, medium was changed and cells were maintained in ITTC medium until day 10. Cells were fixed (paraformaldehyde 4%, 15 min, RT), and stained with Bodipy 493/503 (10 μg/ml, 15 min, Invitrogen) and Hoechst 33342 (5 μg/ml, 15 min, Invitrogen). Stainings were observed by inversed fluorescent microscope (Nikon Eclipse TE300) and 5 pictures were randomly taken under 20X objective (numeric camera and acquisition software NIS-Elements 2.5 BR, Nikon®). Differentiation area (Bodipy stained area, μm²) was measured with ImageJ software and normalized by nuclei cell number in order to obtain the differentiation area (μm²) per cell.

BMP7 adipogenic culture protocol

Progenitor cells were treated during 2 days with recombinant human BMP7 (50 ng/mL) then 9 days in adipogenic medium (ITTC).

MSCA1 gene expression and activity were measured. UCP1 gene expression was measured by RT-PCR.

mRNA extraction and Real-Time PCR.

Total RNAs were extracted using the RNeasy kit (Qiagen). The RNA concentration was measured by fluorimetric assay (Ribogreen, Invitrogen). RNAs were reverse-transcribed using Superscript® II kit (Invitrogen). TaqMan® Primers are listed in supplemental data Table 1. The amplification reaction was done in duplicate on 15 ng of the cDNA samples in a final volume of 20 μL in 96-well reaction plates (Applied Biosystems) in a GeneAmp 7500 detection system (Applied Biosystems). All reactions were performed under the same conditions: 50° C. for 2 minutes, 95° C. for 10 minutes, 40 cycles of 95° C. for 15 seconds, and 60° C. for 1 minute. Results were analyzed with the GeneAmp 7500 software and all values were normalised to 18S rRNA levels.

### Immunofluorescence Staining

Cells or small pieces of whole AT were fixed (paraformaldehyde 4%, room temperature for 15 min for cultured cells and up to 1 h for whole AT). After blocking step (PBS 2% BSA), or blocking and permeabilisation step (PBS, 2% BSA, 0.1% tritonX100) depending on antigen, samples were incubated with primary antibody for 1 h at RT (overnight at 4° C. for whole AT) under shaking. After washing and blocking steps, samples were incubated with appropriate secondary antibodies coupled with Alexa Fluor 488 or 546 (Invitrogen). Nuclei were stained by Hoechst 33342 (Invitrogen).

MSCA1 inhibition

Progenitor cells were cultured under adipogenic conditions with or without 50 μM or 500 μM of tetramisole hydrochloride, an alkaline phosphatase inhibitor. After 3 to 10 days, Bodipy staining was performed or RNAs extracted. Progenitor cells were seeded at the density of 90000/cm2 cells in 12 well-plates. After 24 hours, cells were transfected with 500 ng/mL of MISSION® esiRNA against tissue non-specific alkaline phosphatase (MSCA1) or GFP as negative control with 1 μL of lipofectamine® 2000 (invitrogen) in 1 mL of basal medium. After 12 hours, medium was removed and cells were rinsed once with PBS and cultured under adipogenic conditions for 3 to 10 days and RNAs were extracted.

Subcutaneous injection of human native adipose tissue progenitor cells by fibrin-based delivery

Female NMRI mice (Janvier, Genest St. Isle, France) were anesthetized with isoflurane and subcutaneously injected in dorsal area using an insulin Myjector (Terumo France SA, Guyancourt, France) containing 25 μl of fibrin solution (24 mg/ml) (Tissucol® kit, Baxter, Maurepas, France) mixed with either 1.106 cells of each AT progenitor subsets or no cells (in 25 μl of ECBM) and 50 μl of thrombin solution (5 UI/ml) (Baxter). Each mice was injected with four distinct gels (containing MSCA1+, −/CD271+, −/− or no cells, respectively) randomly implanted at four distinct dorsal positions. Mice were then fed with a high fat diet (45% fat, Research Diet, New Brunswick, USA) for 6 weeks and euthanized. Presence of cells at the gel implantation sites under the skin was then analyzed and when present, cells were fixed (4% paraformaldehyde, 30 min) and processed.

Statistical analyses

Values are given as the mean±sem for (n) independent experiments. Comparisons between groups were analyzed either by two-tailed paired Student's t-test or two-way ANOVA, followed by Bonferroni posttests using Prism (GraphPad Software). Differences were considered statistically significant when P<0.05.

### Results

CD271 and MSCA1 discriminate progenitor cell subsets in human subcutaneous AT

Multiparameter flow cytometry analyses identified three progenitor cell subsets when performed with stroma-vascular fraction (SVF) from subcutaneous abdominal AT and freshly harvested from female donors using CD45, CD34, CD31 CD271 and MSCA1 as markers. Cells belonging to the gated sets are referred to as MSCA1+ (CD45−/CD34+/CD31−/CD271+/MSCA1+), −/CD271+ (CD45−/CD34+/CD31−/CD271+/MSCA1−) and −/− (CD45−/CD34+/CD31−/CD271−/MSCA1−). While CD34/CD31 can separate endothelial versus progenitor cells, further segregation of progenitor cells with CD271/MCSA1 reveals intermediary levels of expression. The MSCA1+ cell subset exhibited the highest mitochondrial content (2-fold more than −/− cells, P<0.05).

The percentage of MSCA1+ cells within the native progenitor cell population was increased in individuals with higher adiposity index, an effect not paralleled by CD271 (3-fold increase in obese compared to lean women). MSCA1 mRNA levels also increased with obesity in native CD45−/CD34+/CD31− cells (4-fold increase). The expression levels of MSCA1, CD271 and two other AT progenitor cell markers ZFP423 and Pref1 were determined by real-time PCR experiments in freshly harvested human mature adipocytes and various immunoselected/depleted cell types from AT SVF, i.e., endothelial cells (defined as CD34+/CD31+), macrophages (defined as CD34−/CD14+) and progenitor cells. Both MSCA1 and CD271 transcripts were predominantly expressed in progenitor cells compared to other cell types. ZFP423 expression was similar in both endothelial and progenitor cells while the expression of Pref1, a classic murine preadipocyte marker, was low to undetectable in 3 out of 6 progenitor cell sample. Immunohistochemical analyses using CD34 and MSCA1 antibodies on whole subcutaneous AT showed that co-labeled cells were predominantly located in the AT stroma, whereas perivascular cells were mainly CD34 positive and MSCA1 negative.

Higher MSCA1+ cell content is associated with stronger adipogenic potential of subcutaneous versus visceral progenitor cells

Multiparameter flow cytometry analyses, performed on the native SVF obtained from matched biopsies of subcutaneous (abdominal) and visceral (omental) AT of obese individuals showed that the percentage of MSCA1+ cells was higher in subcutaneous (n=22, P<0.01). To expand these observations to mice, experiments were performed with AT from C57B16 mice using the murine equivalent Alpl, since MSCA1 was described to be identical to tissue non-specific alkaline phosphatase (Sobiesiak et al., 2010). Alpl discriminates cells into distinct progenitor subsets in the CD45−/CD31−/SCA1+ murine cell population, equivalent to the human CD45−/CD34+/CD31− population. As observed in humans, Alpl/Sca1 flow cytometry profiles were unable to clearly segregate populations due to intermediary expression levels for both Alpl and Sca1. Inguinal subcutaneous AT possessed 16-fold more Alpl+ cells than epididymal AT. The higher MSCA1+ cell content in the progenitor cells of human subcutaneous AT was associated with higher adipogenic capacity compared to visceral AT. In mice, higher Alpl+ content in subcutaneous inguinal AT was associated with a higher number of adipocytes compared to visceral epididymal AT.

MSCA1+ cells originate from the adipogenic commitment of CD45−/CD34+/CD31− cells

The impact of adipogenesis was then studied on both MSCA1 and CD271 transcript levels in human native CD45−/CD34+/CD31− progenitor cells. Transcript levels of MSCA1 were already increased 5-fold after the commitment period (P<0.05, n=6) and remained elevated for the following 7 days. This profile was similar to that observed for the adipogenic transcription factors PPARγ2 and ChREBP, whereas C/EBPa mRNA increased later with terminal differentiation. In contrast, the expression of CD271 was gradually inhibited all along the commitment and differentiation process as observed for the anti-adipogenic genes such as FSP1 (Fibroblast specific protein 1) and DNER (Delta Notch EGF-like receptor). The adipogenic commitment-dependent increase in MSCA1 mRNA levels was associated with enhanced MSCA1 protein expression, as shown by flow cytometry analyses after the commitment period (2.5-fold increase in mean fluorescence intensity, P<0.01, n=13). In parallel, a 5-fold increase in the percentage of MSCA1+ cells was observed (P<0.01).

MSCA1 is involved in adipocyte differentiation

The inventors studied the potential involvement of alkaline phosphatase activity in adipocyte differentiation. Treatment of native human AT progenitor cells in adipogenic culture conditions with tetramisole, an inhibitor of alkaline phosphatase activity, led to a concentration-dependent decrease in adipocyte differentiation without altering the total number of nucleated cells. In agreement with reduced differentiation, the expression of the adipogenic transcription factors PPARγ2, CHREBP and C/EBPα as well as the adipocyte- specific genes LPL, GPDH and PLIN1, were decreased at day 10. No statistically significant effect was observed after the commitment period at day 3. To further demonstrate the involvement of MSCA1 in adipocyte differentiation, native human AT progenitor cells were transfected with esiRNA for MSCA1, or esiRNA-GFP as a control, and cultured under adipogenic conditions. The extent of differentiation was only minimally lowered in progenitor cells transfected with esiRNA-GFP compared to non-transfected cells, while transfection with esiRNA for MSCA1 enabled a 35% inhibition of MSCA1 expression. After 10 days under adipogenic culture conditions, diminished MSCA1 expression was accompanied by a marked reduction in differentiation, as shown by decreased expression of the adipogenic transcription factors PPARγ2, CHREBP and C/EBPα and GPDH, a marker for adipocyte differentiation. In agreement with the differentiation-specific role of MSCA1, no changes in gene expression were observed after the commitment period at day 3.

The native immunoselected MSCA1+ progenitor cell subset possesses the highest expression levels for white and brite/beige adipocyte markers

An immunoselection/depletion approach was developed to selectively isolate the distinct progenitor cell subsets from native stroma-vascular cells of human AT. The purity of each cell subset was controlled by flow cytometry analyses. Hierarchical clustering of gene expression profiles from the three progenitor cell subsets clearly showed that the MSCA1+ cell subset was set apart from the cluster containing −/− and −/CD271+ cells. The expression levels of genes described to be anti-adipogenic (decrease in expression with differentiation), were lower for both MSCA1+ and −/CD271+ cells compared to −/− cells. The mRNA levels for most of the white or brite/beige adipogenic markers were highest in MSCA1+ cells. MSCA1+ cells also exhibited the highest expression level of β-adrenergic receptor 2. As expected for human adipocytes, β-adrenergic receptor 3 expression was undetectable.

The native immunoselected MSCA1+ progenitor cell subset exhibits the highest adipogenic differentiation potential in vitro and in vivo

The impact of distinct adipogenic conditions were compared using whole native progenitor cells and our three subsets based on CD271/MCSA1. These conditions include induction of brown differentiation through treatment with the cAMP elevating compound IBMX and commitment/differentiation with rosiglitazone, a PPARγ agonist. IBMX only slightly and insignificantly increased differentiation in whole progenitor cells. As expected, commitment and differentiation were markedly enhanced with rosiglitazone. MSCA1+ cells exhibited the highest differentiation potential under both conditions. Indeed, MSCA1+ cells were the sole progenitor cell subset to differentiate under IBMX stimulation, as shown by the marked appearance of lipid laden cells. In addition, UCP1-positive cell clusters were only found in cells from the MSCA1+ subset. Under rosiglitazone treatment, each cell subset behaved differently in terms of the commitment/differentiation processes. MSCA1+ cells exhibited the highest number of lipid-laden cells without marked changes in total cell number, a hallmark of increased differentiation. Rosiglitazone treatment of −/CD271+ and −/− cells induced both adipogenic commitment and differentiation, as shown by an increase in total cell number combined with a moderate enhancement in lipid-laden cells. Real time PCR analyses showed increased expression of white and brite/beige adipocyte-related genes in the MSCA1+ cell subset, compared to the other progenitor cell subsets, 3 days after exposure to adipogenic medium containing rosiglitazone. Identical results were obtained using our cell sorting approach to isolate distinct progenitor cell subsets by flow cytometry.

BMP7 treatment (at 50 ng/mL for 2 days) significantly increased the expression and the activity of the MSCA1 gene and led to a significant increase in UCP1 gene expression, indicating a differentiation towards brite adipocytes.

Finally, to address the question of “in vivo” adipogenic potentials of human progenitor cell subsets, female NMRI mice were injected subcutaneously with fibrin gels containing human native immunoselected MSCA1+, −/CD271+and −/− cell subsets (n=3). After six weeks under high fat feeding, the subcutaneous implantation sites were removed and analyzed. Implantation sites from MSCA1+ containing gels were characterized by marked adipogenesis and implantation sites from −/CD271+and −/− containing gels exhibited few if any adipocytes. MSCA1+ cells were able to develop a fat pad despite being isolated from crude AT-SVF and not undergoing any challenge.

### Conclusion

The inventors have shown that the progenitor cell population is heterogeneous in terms of expression of two mesenchymal stem/stromal cell markers, CD271 (p75 neurotrophin receptor (NGFR)) and MSCA1 (mesenchymal stem cell antigen 1 also known as tissue non-specific alkaline phosphatase (ALPL)) (Sobiesiak et al., 2010) (Battula et al., 2009). Three cell subsets were identified in freshly harvested, native human CD34+/CD31− cell populations: CD34+/CD31−/CD271+/MSCA1+, CD34+/CD31−/CD271+/MSCA1−, and CD34+/CD31−/CD271−/MSCA1− (referred to as MSCA1+, −/CD271+and −/−, respectively). Whereas immunofluorescence assay shows that the percentage of UCP1 positive cells is 25,7±34,6% when native progenitors are CD45−/CD34+/CD31−, this percentage is of 82,8±12,2% when CD45−/CD34+/CD31−/CD271+/MSCA1+ are selected and 22,5±10,2% for CD45−/CD34+/CD31−/CD271+/MSCA1− and 17,0±8,7% for CD45−/CD34+/CD31−/CD271−/MSCA1−.

The inventors have shown that the MSCA1+ cell subset is predominantly found in stromal locations and exhibits a high mitochondrial content. In human subcutaneous AT, MSCA1+ cells are increased with obesity and show an AT-location dependent expression, with higher levels in subcutaneous AT over visceral AT. They have also demonstrated that MSCA1 is an early marker of adipogenesis and that pharmacological or molecular interference of MSCA1 activity inhibits adipogenesis. By independently isolating the three distinct sub populations through either immunoselection/depletion or cell sorting, they have found that MSCA1+ cells exhibit the highest white and brite/beige adipogenic capacities both in vitro and in vivo.

In conclusion, MSCA1 represents a human AT marker for common white and brite/beige preadipocytes. MSCA1 represents a useful tool for tracing dynamic changes in brite/beige preadipocytes in various pathological and/or experimental conditions in humans.

### REFERENCES

Barbatelli G, Murano I, Madsen L, Hao Q, Jimenez M, Kristiansen K, Giacobino JP, De Matteis R, and Cinti S. The emergence of cold-induced brown adipocytes in mouse white fat depots is determined predominantly by white to brown adipocyte transdifferentiation. Am J Physiol Endocrinol Metab 298: E1244-1253, 2010.

Battula VL, Treml S, Bareiss PM, Gieseke F, Roelofs H, de Zwart P, Muller I, Schewe B, Skutella T, Fibbe WE, Kanz L, and Buhring HJ. Isolation of functionally distinct mesenchymal stem cell subsets using antibodies against CD56, CD271, and mesenchymal stem cell antigen-1. Haematologica 94: 173-184, 2009.

Cinti S. Between brown and white: novel aspects of adipocyte differentiation. Ann Med 43: 104-115, 2011.

Cristancho AG and Lazar MA. Forming functional fat: a growing understanding of adipocyte differentiation. Nat Rev Mol Cell Biol 12: 722-734, 2011.

Gburcik V, Cawthorn WP, Nedergaard J, Timmons JA, and Cannon B. An essential role for Tbx15 in the differentiation of brown and “brite” but not white adipocytes. Am J Physiol Endocrinol Metab 303: E1053-1060, 2012.

Gesta S, Tseng YH, and Kahn CR. Developmental origin of fat: tracking obesity to its source. Cell 131: 242-256, 2007.

Gupta RK, Arany Z, Seale P, Mepani RJ, Ye L, Conroe HM, Roby YA, Kulaga H, Reed RR, and Spiegelman BM. Transcriptional control of preadipocyte determination by Zfp423. Nature 464: 619-623, 2010.

Ishibashi J and Seale P. Medicine. Beige can be slimming. Science 328: 1113-1114, 2010.

Lafontan and Girard 2008 Impact of visceral adipose tissue on liver metabolism. Part I: heterogeneity of adipose tissue and functional properties of visceral adipose tissue. Diabetes Metab. 2008 Sep;34(4 Pt 1):317-27

Lee YH, Petkova AP, Mottillo EP, and Granneman JG. In vivo identification of bipotential adipocyte progenitors recruited by beta3-adrenoceptor activation and high-fat feeding. Cell Metab 15: 480-491, 2012.

Macotela Y, Emanuelli B, Mori MA, Gesta S, Schulz TJ, Tseng YH, and Kahn CR. Intrinsic differences in adipocyte precursor cells from different white fat depots. Diabetes 61: 1691-1699, 2012.

Maumus M, Peyrafitte JA, D'Angelo R, Fournier-Wirth C, Bouloumie A, Casteilla L, Sengenes C, and Bourin P. Native human adipose stromal cells: localization, morphology and phenotype. Int J Obes (Lond), 2011.

Miranville A, Heeschen C, Sengenes C, Curat CA, Busse R, and Bouloumie A. Improvement of postnatal neovascularization by human adipose tissue-derived stem cells. Circulation 110: 349-355, 2004.

Petrovic N, Walden TB, Shabalina IG, Timmons JA, Cannon B, and Nedergaard J. Chronic peroxisome proliferator-activated receptor gamma (PPARgamma) activation of epididymally derived white adipocyte cultures reveals a population of thermogenically competent, UCP1-containing adipocytes molecularly distinct from classic brown adipocytes. J Biol Chem 285: 7153-7164, 2010.

Rodeheffer MS, Birsoy K, and Friedman JM. Identification of White Adipocyte Progenitor Cells In Vivo. Cell, 2008.

Seale P, Bjork B, Yang W, Kajimura S, Chin S, Kuang S, Scime A, Devarakonda S, Conroe HM, Erdjument-Bromage H, Tempst P, Rudnicki MA, Beier DR, and Spiegelman BM. PRDM16 controls a brown fat/skeletal muscle switch. Nature 454: 961-967, 2008.

Seale P, Conroe HM, Estall J, Kajimura S, Frontini A, Ishibashi J, Cohen P, Cinti S, and Spiegelman BM. Prdm16 determines the thermogenic program of subcutaneous white adipose tissue in mice. J Clin Invest 121: 96-105, 2011.

Sengenes C, Lolmede K, Zakaroff-Girard A, Busse R, and Bouloumie A. Preadipocytes in the human subcutaneous adipose tissue display distinct features from the adult mesenchymal and hematopoietic stem cells. J Cell Physiol 205: 114-122, 2005.

Smas CM and Sul HS. Pref-1, a protein containing EGF-like repeats, inhibits adipocyte differentiation. Cell 73: 725-734, 1993.

Sobiesiak M, Sivasubramaniyan K, Hermann C, Tan C, Orgel M, Treml S, Cerabona F, de Zwart P, Ochs U, Muller CA, Gargett CE, Kalbacher H, and Buhring HJ. The mesenchymal stem cell antigen MSCA-1 is identical to tissue non-specific alkaline phosphatase. Stem Cells Dev 19: 669-677, 2010.

Spalding KL, Amer E, Westermark PO, Bernard S, Buchholz BA, Bergmann O, Blomqvist L, Hoffstedt J, Naslund E, Britton T, Concha H, Hassan M, Ryden M, Frisen J, and Amer P. Dynamics of fat cell turnover in humans. Nature 453: 783-787, 2008.

Tang QQ and Lane MD. Adipogenesis: from stem cell to adipocyte. Annu Rev Biochem 81: 715-736, 2012.

Tang W, Zeve D, Suh J, Bosnakovski D, Kyba M, Hammer B, Tallquist MD, and Graff JM. White Fat Progenitor Cells Reside in the Adipose Vasculature. Science, 2008.

1. A method for obtaining a population brite/white preadipocytes from precursor cells comprising the step of selecting cells for the expression of mesenchymal stem cell antigen 1 (MSCA1).

2. The method according to claim 1, wherein said precursor cells are adipose tissue stroma-vascular cells.

3. The method according to claim 1, wherein the step of selecting cells for the expression of MSCA1 is carried out by immunoselection.

4. The method according to claim 3, wherein the step of selecting cells for the expression of MSCA1 is carried out by immunoselection using magnetic beads or Fluorescence-Associated Cell Sorting (FACS).

5. The method according to claim 1, further comprising a step of immortalizing the MSCA1-positive cells.

6. An isolated population of brite/white preadipocytes obtained by selecting cells for the expression of mesenchymal stem cell antigen 1 (MSCA1).

7. (canceled)

8. A method for screening drugs useful for treating obesity comprising:
obtaining a population of brite/white preadipocytes by selecting cells for the expression of mesenchymal stem cell antigen 1 (MSCA1);
incubating said cells in the presence of a test compound;
assessing brite adipogenesis; and
concluding that said test compound is a drug useful for treating obesity if the number of brite adipocytes obtained after incubation with the test compound is higher than the number of brite adipocytes obtained after incubation in the absence of the test compound.

9. A method for obtaining an isolated population of brite adipocytes comprising:
obtaining a population of brite/white preadipocytes by selecting cells for the expression of mesenchymal stem cell antigen 1 (MSCA1);
culturing said brite/white preadipocytes in the presence of an agent that induces brite adipogenesis.

10. The method according to claim 9 wherein the agent that induces brite adipogenesis is selected from the group consisting of IBMX, BMP7 and mixtures thereof.

11. A population of isolated brite adipocytes obtained by
obtaining a population of brite/white preadipocytes by selecting cells for the expression of mesenchymal stem cell antigen 1 (MSCA1);
culturing said brite/white preadipocytes in the presence of an agent that induces brite adipogenesis.

12. (canceled)

13. A method of treatment of obesity in a subject in need thereof, comprising
administering to said subject an efficacious amount of a substantially pure homogenous population of brite adipocytes.

14. A method for screening drugs useful for treating obesity comprising:
obtaining a population of brite preadipocytes by selecting cells for the expression of mesenchymal stem cell antigen 1 (MSCA1);
culturing said brite/white preadipocytes in the presence of an agent that induces brite adipogenesis to form an isolated population of brite adipocytes
incubating said brite adipocytes in the presence of a test compound,
assessing adipogenesis, and
concluding that said test compound is a drug useful for treating obesity if the number of brite adipocytes obtained after incubation with the test compound is higher than the number of brite adipocytes obtained after incubation in the absence of the test compound.

15. (canceled)
